(19) United States
(12) Patent Application Publication
(10) Pub. No.: US 2017/0043086 A1
(43) Pub. Date: Feb. 16, 2017

(54) ENHANCED CANNULATION METHOD AND NEEDLES

(71) Applicant: Liviu Popa-Simil, Los Alamos, MN (US)

(72) Inventor: Liviu Popa-Simil, Los Alamos, MN (US)

(21) Appl. No.: 14/164,624

(22) Filed: Jan. 27, 2014

Related U.S. Application Data
(60) Provisional application No. 61/761,386, filed on Feb. 6, 2013.

(51) Int. Cl. A61M 5/158; A61M 1/36; A61M 5/172

(52) U.S. Cl. CPC A61M 5/158; A61M 5/1723; A61M 1/3653; A61M 2005/1586

(57) ABSTRACT

A novel set of flexible dialysis tube, needles, tubing and related attachments that may be used to improve the blood sampling, removal, and reinfusion process and reduce the medical hazards of such procedures for the patient. It consists in a special perforation needle over which a plastic tube is passed into the blood vessel, flexible dialysis tube remains inserted and opens gently then bends and morphs to the body structure assuring a good blood flow. A version of flexible dialysis tube may have inside valves and actuators so that bi-directional flow can be obtained following a single vessel puncture instead of two, for procedures such as hemodialysis. An exterior connection box allows a patient to connect to an external blood processing device (hemodialysis) quickly and safely. Further, the device is designed to stay in place for several days or more, further reducing the risks and discomforts of repeated vessel punctures. Some versions of the device could have a micro-sensor array embedded with the plastic tube, enabling continuous measurement of many medically significant parameters.

### CROSS REFERENCE TO RELATED APPLICATIONS

This application claims the benefit of U.S. Provisional Application No. 61,761,386 from Feb. 6, 2013 and NO International application.

### BACKGROUND OF THE INVENTION

1. Field of the Invention

The present invention relates to a method and devices to increase the comfort and safety of intravascular access and perfusion (removal and/or reinfusion of blood or other fluid), in order to minimize its negative impact for the patient, reduce the time required for such procedures, and improve the quality of such procedures in numerous ways.

Many medical procedures require prolonged or repeated large-bore intravascular access for infusion of drugs, parenteral nutrition, and hemodialysis. The present method and devices come to improve the process of vessel penetration, and the compatibility of the devices with the blood vessels and blood, among other benefits.

For the many procedures that require repetitive access to the patient's blood vessels, such as hemodialysis, this method reduces the frequency of vessel perforations that are required. By reducing vessel trauma, these method and devices may reduce the many dangerous medical complications and expensive surgical interventions, suffered by dialysis and similar patients. These devices may also serve as a safe long-lived blood-port with capabilities of monitoring the perfusion process and gathering other physiologic data.

2. Description of the Prior Art

Historically, kidney diseases have been a major concern among human diseases. When the kidney is sufficiently impaired that a large fraction of the body's waste products and water are not removed from the blood, the life of the patient cannot be preserved unless means are provided for artificially performing the function of the impaired kidney. Various processes called “dialysis” are used to remove these waste products.

The most commonly accepted practice for dialyzing a patient's blood extracorporeally requires the surgical creation of a subcutaneous, arterial-venous fistula—a conduit, also called a shunt, for a flow of blood from an artery, usually in an arm, to a vein.

Thereafter, a relatively large flow of blood produces dilation of the subcutaneous venous system, giving sufficient blood flow for dialysis by venipuncture of this “shunt” with large bore needles.

Normally, two hollow needles or cannulas are used to perform two venipunctures into the shunt, so that blood can be simultaneously withdrawn and (purified blood) reinfused.

Conventionally, blood is withdrawn from one of the needles, pumped through a hemodialyzer and thereafter pumped back into the patient. The needles have to be substantially distant from one another to prevent recirculation of blood.

The aforementioned methodology has been found to have serious disadvantages both to the patient and to the attending physicians, nurses, and technicians. The problems are particularly aggravated because most patients requiring extracorporeal hemodialysis must undergo treatment as frequently as three to four times per week. This means that if every venipuncture were completely successful, a patient 50 would need to undergo from 6 to 8 venipunctures or cannulations each week.

It is well-known that the lifespan and proper function of a fistula is inversely related to the number of venipunctures. Shunts that are repeatedly subjected to the 55 trauma of venipuncture are much more susceptible to thrombophlebitis, perivascular hemorrhage, clotting and infection. In fact, it is commonly found in patients who have experienced a number of venipunctures, that the tissues surrounding the most accessible veins develop large hematomas which obscure the veins, making successful venipuncture extremely difficult.

Also contributing to the problem is the fact that once one successful venipuncture is made and blood is allowed to 65 flow from the patient's body toward a hemodialyzer, the blood volume in the fistula is reduced, making the second venipuncture very difficult. It has historically been found that while most skilled physicians or technicians are able to perform the first venipuncture with little difficulty, frequently numerous attempts are necessary before a 5 second venipuncture can be performed.

In addition, the multiple attempts at venipuncture often necessary to place the second needle result in worsening apprehension and anxiety on the part of both the patient and the physician, nurse, or technician attending the patient further reducing the likelihood of successful venipuncture.

In order to access the blood vessels for dialysis, perfusion or other purposes, it is first necessary to penetrate the blood vessel by puncturing it with a needle, and then, if the needle itself is not to remain in place, inserting a flexible tube of some kind, most often using the lumen of the same needle used to puncture and penetrate the blood vessel. In some devices, the flexible tube covers the needle as a sheath, and remains in the vessel after the inserting needle is removed.

Dialysis typically uses a special cannulation technique that requires two punctures; one up-stream collecting arterial blood entering the shunt, and another downstream, near the venous end of the shunt, for return of the purified blood.

There are several existing cannulation techniques that use sharp or blunt AV Fistula or button hole needles, that when used repetitively may cause severe blood vessel damage (aneurysm, etc.) requiring medical intervention.

U.S. Pat. No. 4,936,835 describes an improved needle which has a bio-absorbable gelatin cutting or puncturing tip. The gelatin's characteristic renders the needle incapable of penetration after one initial use. Additionally, the gelatin partially dissolves to leave a coating on the punctured tissue margin, which acts to minimize hemorrhaging complications. A non-bioabsorbable in-situ sheath positioned at the punctured tissue site, which compresses the tissue, alternatively addresses hemorrhaging complications. This system has the potential problems of reaction to the small amounts of chemicals introduced, as well as complications from the solid steel needle damaging the inside of the fistula, that are prevented by the present invention

U.S. Pat. No. 6,962,575 82 from Nov. 8, 2005 discloses a single access dialysis needle system comprising a first cannula, a second cannula or sheath, and a barrier arranged on the outer surface of the first cannula. The distal end of the first cannula extends distal to the distal end of the second cannula or outer sheath, and the barrier is positioned between the respective distal ends. When the barrier is inflated or otherwise activated, it prevents or minimizes recirculation. This procedure has the disadvantage of a much bigger hole penetrating the fistula, and more distress to the interior of the fistula, from the two tubes and the inflated sealing barrier that are removed by the present patent.

The patent US20080312577 teaches a veno-venous expandable dialysis apparatus including a blood injection needle component configured to introduce blood at a position of a first peripheral vein and a blood withdrawal needle component configured to withdraw blood at another position from a second peripheral vein, where the first position is located away from the second position. The expandable dialysis apparatus further includes a guide wire having a central axis, an expanding sheath configured circumferentially around the guide wire to form an annular lumen between a distal blood withdrawal position and a proximal extracorporeal position; and a needle disposed around the expandable sheath. The patient is still harmed by the presence of the guiding wires, as well as by the stiffness of the needles and repetition of the puncturing; these inconveniences are eliminated by the present invention, too.

The book written by Kaufman J A, Lee M J. On “Vascular and Interventional Radiology. The Requisites” in 2004 disclosed a procedure for the placement of a dialysis catheter. They clearly stated that a strict aseptic technique must be used during insertion procedure. Chronic dialysis catheters (CDCs) are cuffed, tunneled catheters. The configuration is dual-lumen, with an arterial port for blood flow from the body, and a venous port for blood return after passing through the dialysis machine. Risk of recirculation of blood is decreased by a staggered tip design.

Flow rate is an important consideration in tunneled CDC design, as faster flow rates decrease dialysis time for the patient, and this is another aspect improved by the present invention.

Generally, for tunneled CDCs, the preferred veins for central access are the right internal jugular (RIJ), right external jugular (REJ), left internal jugular (LIJ), left external jugular (LEJ)—in that order.

The National Kidney Foundation's Kidney Disease Outcomes Quality Initiative Clinical Practice Guidelines for Hemodialysis Adequacy (K/DOQI Guidelines) state that subclavian vein (SCV) catheterization should be avoided in patients with end stage renal disease (ESRD) because of the risk for central venous stenosis, with subsequent loss of the entire ipsilateral arm for vascular access.

The tunnel for the CDC is created by advancement of a tunneling device through the subcutaneous tissue on the chest wall. The tunnel may be placed medially, with the exit site at a parasternal infra-clavicular location. Alternately, it may be placed laterally, with the exit site below the clavicle at the delto-pectoral groove. The cuff of the tunneled CDC acts to hold the catheter in place. In addition, it is designed to cause a fibrotic reaction, creating a physical barrier to bacteria that prevents bacterial migration and inoculation via the exit site. The cuff is positioned within the tunnel at a distance from the exit site that will facilitate removal.

The present invention may use a catheter inserted in a peripheral arterio-venous shunt or large central vein as a chronic dialysis catheter, aiming to reduce the several risks associated with the existing chronic dialysis catheter systems.

Hemodialysis often involves fluid removal (using ultrafiltration in the dialysis machine), because most patients with renal failure pass little or no urine and accumulate excess intravascular volume. Side effects caused by removing too much fluid and/or removing it too rapidly include low blood pressure, fatigue, chest pains, leg-cramps, nausea and headaches. These symptoms can occur during the treatment and can persist post-treatment; they are sometimes collectively referred to as the dialysis hangover or dialysis washout The severity of these symptoms is usually proportional to the amount and speed of fluid removal. However, the impact of a given amount or rate of fluid removal can vary greatly from person to person and day to day. These side effects can be avoided and/or their severity lessened by limiting fluid intake between treatments or increasing the intensity of dialysis e.g. dialyzing more often or longer per treatment than the standard three times a week, 3-4 hours per treatment schedule. The present invention limits these side effects by allowing continuous monitoring of patients' parameters, using the embedded electronics, and keeping the flow rate optimal.

Since hemodialysis requires access to the circulatory system, patients undergoing hemodialysis may have their blood exposed to microbes, which can lead to sepsis (infection in the blood), endocarditis, (infection on the heart valves), or osteomyelitis, (an infection within the bones). The risk of infection depends on the type of access used and many other variables. Bleeding may also occur at the access sites, again the risk varies depending on the type of access used. Infections can be minimized by strictly adhering to infection control best practices, another goal which the present patent facilitates.

Daily hemodialysis is typically used by those patients who do their own dialysis at home. It is less physiologically stressful, but does require more frequent vessel access. Home hemodialysis is usually done for 2 hours at a time, six days a week. This is simple with indwelling chronic catheters, but more problematic with fistulas or grafts. The “buttonhole technique” can be used for fistulas requiring frequent access. The present invention reduces the inconvenience of repeated vascular puncture, making the patient's home procedure faster and safer.

### SUMMARY

A novel flexible dialysis tube, conceived to minimize the effects of tissue penetration, uses a combination of blood vessel friendly materials, inflicting minimal damage and being designed for multiple uses, with the possibility of remaining installed in the patient's body as a reusable plug-in fixture. In this case a connection box is added that, when activated, seals the blood vessel and cleans the tubes' interior using a fluid-actuator. The connection box creates an antiseptic environment using a combination of chemical and radioactive (alpha and/or beta) active surfaces.

An arterial-venous fistula needle that is used for puncturing the blood vessel having an optimized profile with a narrow cutting edge and a blunt end used to stretch the vessel with minimal cut damage, covered in a bio-compatible light plastic material that creates the tubing connection to the external dialysis or perfusion system. After the penetration is done, needle withdraws, making the flexible dialysis tube's ends open like an umbrella inside the blood vessel providing a good leak-free connection. For dialysis, two puncture points are made that provide a symmetric positioning of the flexible dialysis tubes. For the patient's comfort the plastic flexible dialysis tube bends inside and outside forming a “dog-leg” connection that applies minimum stretching on the nearby tissue and blood-vessel. If properly sealed, it may be maintained for long periods in the patient's body, preventing extra punctures. The long-term use fittings have a special insert that seals the tube and removes any extra blood remaining in the tube, to prevent any infection or static blood deterioration. The outside fixture is equipped with a sterile cover and body that make it safe for long term use.

In order to further minimize the trauma to the patient, it is possible to use a special two in one type of flexible dialysis tubing that pumps the blood intake and blood output through two lumens within the one tube. To enable leaving flexible dialysis tube in the patient's body for long periods, it will contain the additional cleaning and sealing system as well as the sterile cover outside. It will also contain an actuator that will switch the blood flow from the tubes to a shortcut inside the tube inserted in the blood vessel. This technique is superior to the button hole AV Fistula cannulation method because it produces less trauma to the shunt, and reduces drastically the number of medical complications due to shunt deterioration.

### BRIEF DESCRIPTION OF THE DRAWINGS

FIG. 1—Present dialysis needle, longitudinal section

FIG. 2—New AV Fistula needle's cutting end coated in flexible dialysis tube and inserted in a blood vessel

FIG. 3A—New flexible dialysis tube inserted in a blood vessel in intake position and bent into the “dog-leg” position

FIG. 3B—New flexible dialysis tube inserted in a blood vessel in exhaust position and bent into the “dog-leg” position

FIG. 4A—Cross section of the long-term AV Fistula flexible dialysis tube with blood locking and cleaning fixtures,

FIG. 4B—The sterile protection enclosure and flexible dialysis tube function control system.

FIG. 5A—Longitudinal section of the “two in one” cannulation flexible dialysis tube.

FIG. 5B—Cross section of the “two in one” cannulation flexible dialysis tube in AA′.

FIG. 6A—Longitudinal section of the “two in one” long term cannulation flexible dialysis tube.

FIG. 6B—Cross section of the “two in one” long tem cannulation flexible dialysis tube in AA′.

FIG. 7—The sterile protection enclosure and functions control for the “two-in one” flexible dialysis tube.

### DETAILED DESCRIPTION OF THE INVENTION

The inventors consider that most of the problems generated by the actual dialysis and perfusion operations are due to the bad matching between the patient's tissue and the penetration tool, and therefore we develop a new method of penetration, with a more advanced tool, that uses a sharp, stiff needle inside, sheathed outside with a flexible dialysis tube with variable stiffness in harmony with the local function performed, that may bend inside the area to reduce the stress and tissue stretching as much as possible. The assembly comes as the present AV Fistula needle, and in the simplest method is used to implant two needles: one for blood outtake and one for blood inlet.

The penetration is done using the steel needle, then the needle is withdrawn leaving inside a flexible dialysis tube that is bent to accommodate the patient's body, minimizing distress. If many sessions are needed, the sterile connection box may be installed over the area, in order to provide mechanical and bacteriological protection, being an antiseptic enclosure.

In this case flexible dialysis tube has a more complex structure, that includes an inner balloon that inflates with liquid inside, blocking the blood to come out while opening a bypass valve inside the blood vessel switching the flow from the tube outside the body, to straight ahead along the blood vessel, This process of closing the tubing is made using a controlled back flow of sterile liquid, i.e. physiological serum, or equivalent to push the blood out of tube; then the inner balloon inflates blocking the tube.

This operation assures that no residual blood remains in the tube in the interval between consecutive usages of the tube. There are some fine details that have been considered, for example when a flexible dialysis tube, resembling a plastic straw, is entering in a blood vessel against the blood flow it opens a nozzle inside that is made from stiff structure connected by soft structure that makes a tight contact with the blood vessel walls preventing any leak around it. In flexible dialysis tube opening there is an electronic sensor array that can measure the blood pressure, flow rate, pH and even the blood composition and for continuous monitoring purposes. In the case of two needles, we use one to take out blood and one to reintroduce it, leaving a gap between the needles penetration locations. A smaller bypass valve opens in order to allow some blood flow and oxygenate the tissue between the needles, maintaining the blood fresh in the segment of tube between the two flexible dialysis tubes. The exit tube has a softer parachute-like opening that prevents back flow and leakage along the blood vessel perforation.

The presence of sensors inside the blood tube makes possible the real time control of the patient's pressure and the amount of fluid extracted so that post dialysis symptoms could be minimized. The sterile connection box makes possible and easy, safe and fast connection to the patient, also giving electric and optical connection to the monitoring and process instrumentation. One more step forward is reducing the number of perforations from two to one, making the blood extraction and return through the same vessel perforation. This flexible dialysis tube has a more complex structure and is developed in a single connection box. The presence of standardized connectors makes its use safe and comfortable. It also has the facility in emergency cases to be completely pulled off without leaving anything inside the blood vessel and allow the coagulation to seal the wall perforation.

### Best Mode of the Invention

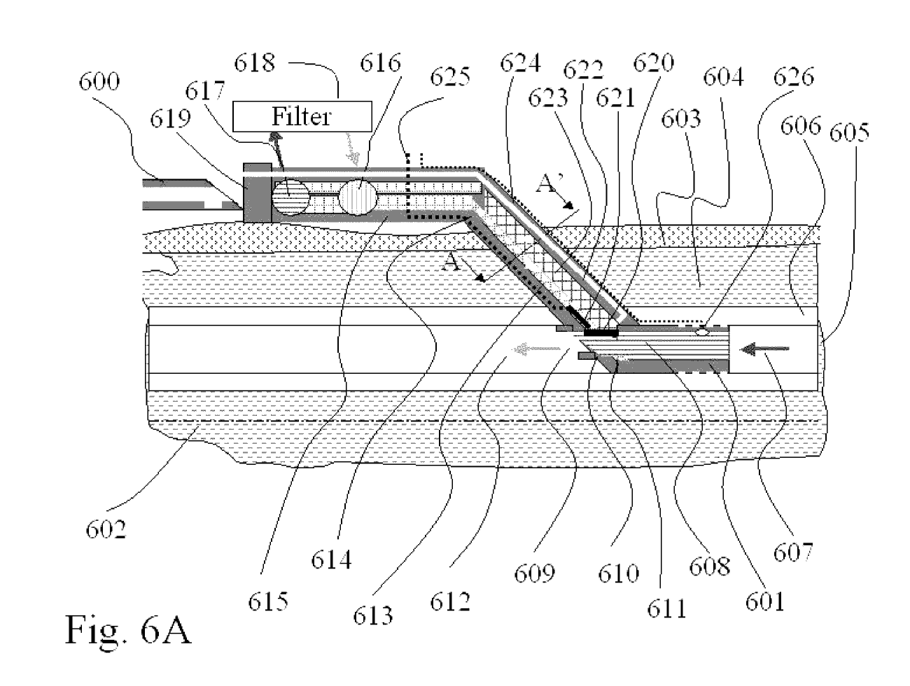

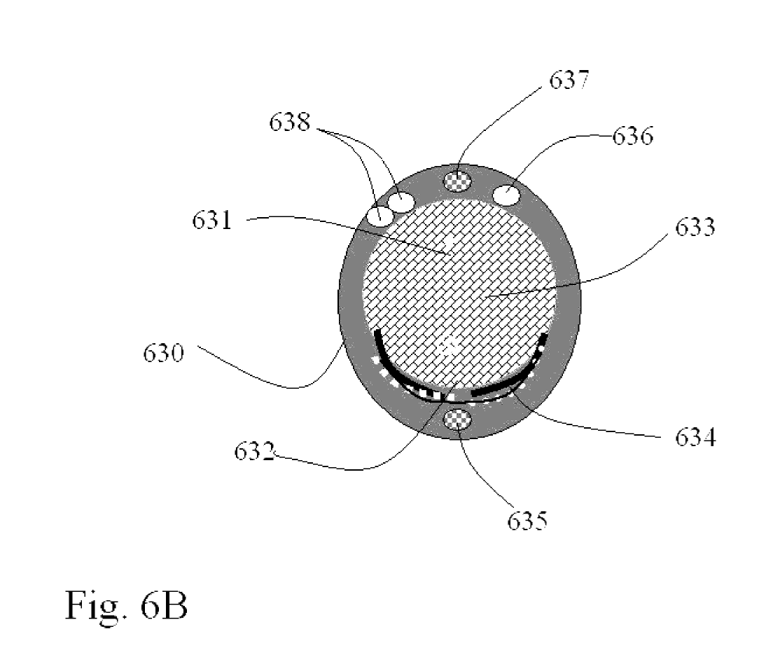

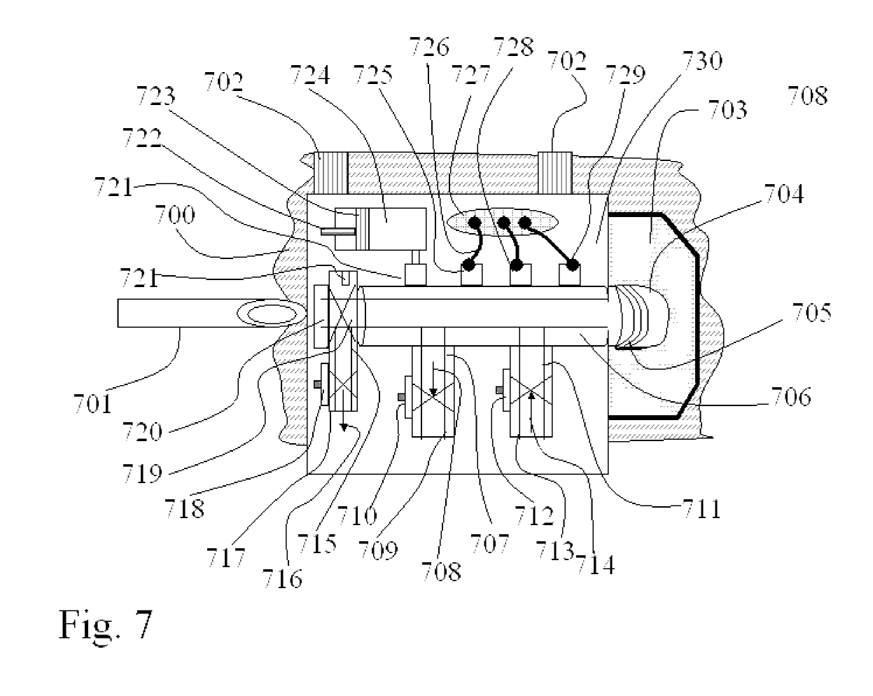

FIGS. 6 and 7 shows the best mode contemplated by the inventors of a 2 in 1 AV Fistula flexible dialysis tube that has improved features. It may be bent to follow the local tissue particularities and minimize their stretching. It opens like an umbrella where the edges are opening against the flow in order to make a tight connection and prevent blood backflow or leakage. In both sides it contains micro-electronic arrays of sensors that are performing blood parameter measurements, to control in real time the equipment operation. As an example, the blood pressure indication may be used to regulate the dialysis pump for optimal perfusion. Using variable in and out pumping, the efficiency of dialysis may be improved, possibly shortening the dialysis time. It also allows the equipment to know how much fluid volume to extract to achieve the optimum volume status for the patient.

The application of a connector box over flexible dialysis tube allows fast connection, safe switching of flexible dialysis tube from off position between sessions to on position and back, and the connection of the desired instrumentation to monitor the patient in real time between sessions and control the equipment operation during the session. By its nature this soft tubing, compatible with the nearby tissue, reduces drastically the thrombosis hazard and other medical complications that require complex surgical intervention.

### How to Make the Invention

As can be seen from the drawings, flexible dialysis tube improvement is the first significant advance. It starts with a smaller steel needle, with an edge partially sharpened in the tip to perforate the tissue and blood vessel, and partially with blunt rounded edge to be used to stretch the blood vessel, without piercing, in order to accelerate recovery. It will have a shoulder to protect the plastic sheathing and facilitate its penetration into the blood vessel. After the sheathing representing flexible dialysis tube penetrates inside the shunt itself, the needle is withdrawn, breaking the seals holding the entry “parachute” closed and makes it open as an umbrella in the tube, sealing on its walls. When the steel needle is withdrawn the bypass valve opens automatically and the backwards umbrella opens creating the secondary seal against the walls of the shunt. The structure is fabricated by making the profiled needle first, and then adding the plastic sheathing, that is made from polymers compatible with body tissue.

Shape remembering polymers may also be used, and make them open over a certain triggering temperature, when they are warmed up by the body heat. The “umbrella” structure is achieved by pressure molding in a die that forms them in open position. Different polymers could be used to form variously rigid parts. It may also be made by fusing together primary assemblies with heat. Tubing for the various actuators and micro-cables will be put in position during the die casting process. After the plastic tubing is mounted tightly on the needle the needle end is added and sealed with the needle, and the needle is installed on the delivery box in aseptic conditions. Chemical treatment with anticoagulants inside and coagulants outside has to be performed before and after flexible dialysis tube is mounted on the needle when access to the surfaces is possible.

The connection box will be delivered in modular parts that are sequentially installed on the patient's body. It contains standardized fast coupling devices for hydraulic actuators as well for the electronic sensor system. It is treated with aseptic materials that prevent bacteria growth and is sealed, possibly using pressurized argon or sterile air.

There will be several types of flexible dialysis tube developed in order to meet the needs of the applications with two or one blood vessel perforation and for long term use with connector box or for immediate use without connector box, everything being in a modular structure that allows cost optimization also.

### DETAILED DESCRIPTION OF THE DRAWINGS

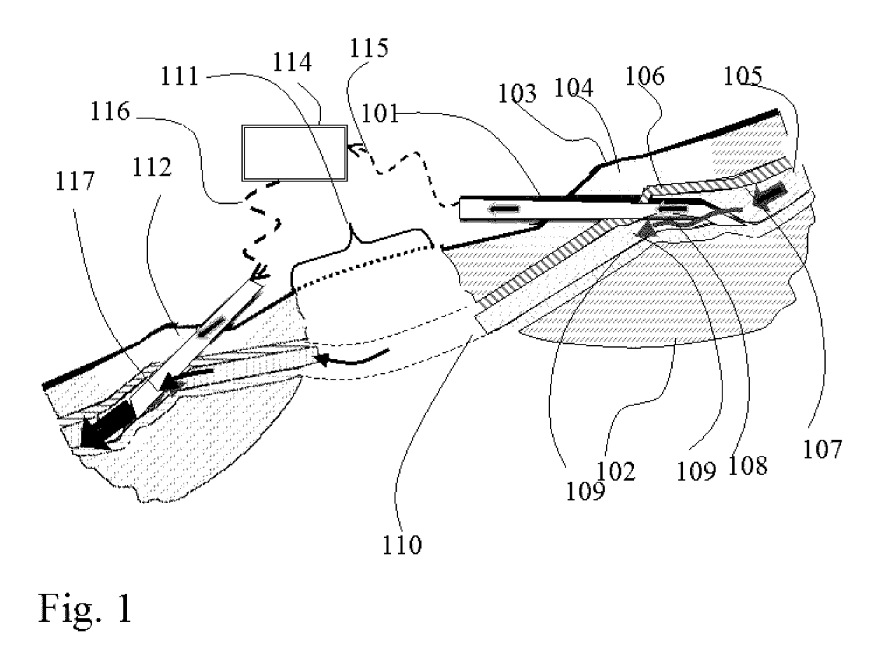

FIG. 1 Present dialysis needle, longitudinal section as it penetrates tissue and a blood vessel.

101—The AV Fistula needle

102—Tissue

103—Skin

104—Sub-cutaneous tissue

105—Blood vessel

106—Blood vessel wall

107—Blood flow

108—Blood flow passing outward through the needle

109—Blood flow passing through the needle bypass hole

110—Residual blood flow past the needle in the vein

111—gap between needles.

112—Second needle that puts blood back in the vein

114—External device (dialysis or analysis)

115—Device's input tube

116—Device's output tube returns to vein

117—Blood flows recombination point

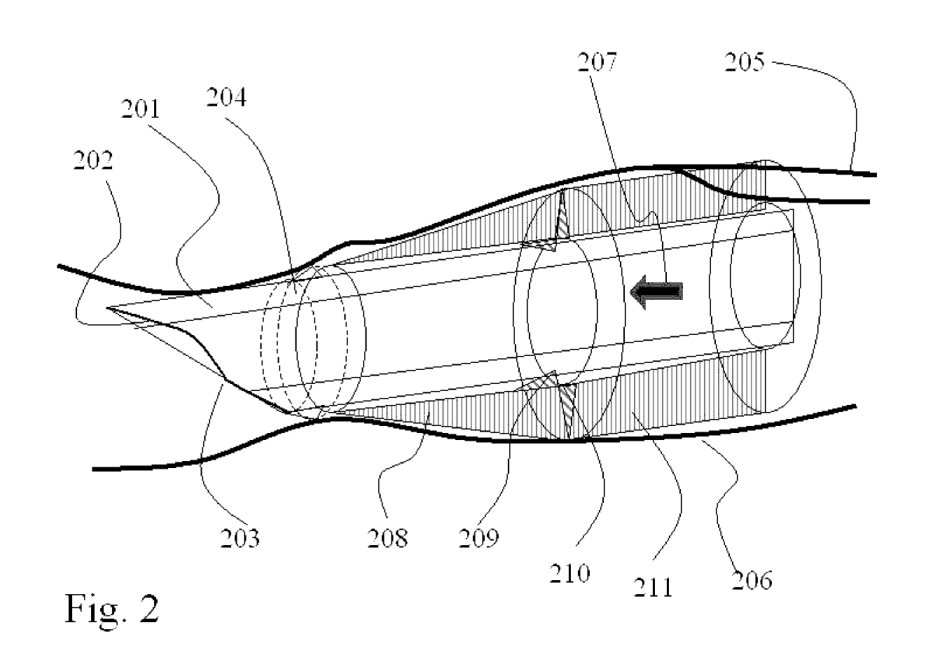

FIG. 2—New AV Fistula flexible dialysis tube needle's cutting end coated in the plastic tubing and inserted in a blood vessel

201—The AV Fistula needle

202—Profiled cutting edge

203—Blunt edge for elastic stretching the blood vessel's hole without cutting

204—Needle bump for plastic flexible dialysis tube umbrella opener activation

205—Fistula penetration hole borders

206—Blood vessel tube wall

207—Blood flow

208—Umbrella structure opening inside blood vessel

209—Umbrella structure opening inside blood vessel bump for triggering opening

210—Umbrella structure opening inside blood vessel with hinge-like structure to allow elastic opening against wall of fistula.

211—Bio-compatible plastic flexible dialysis tube.

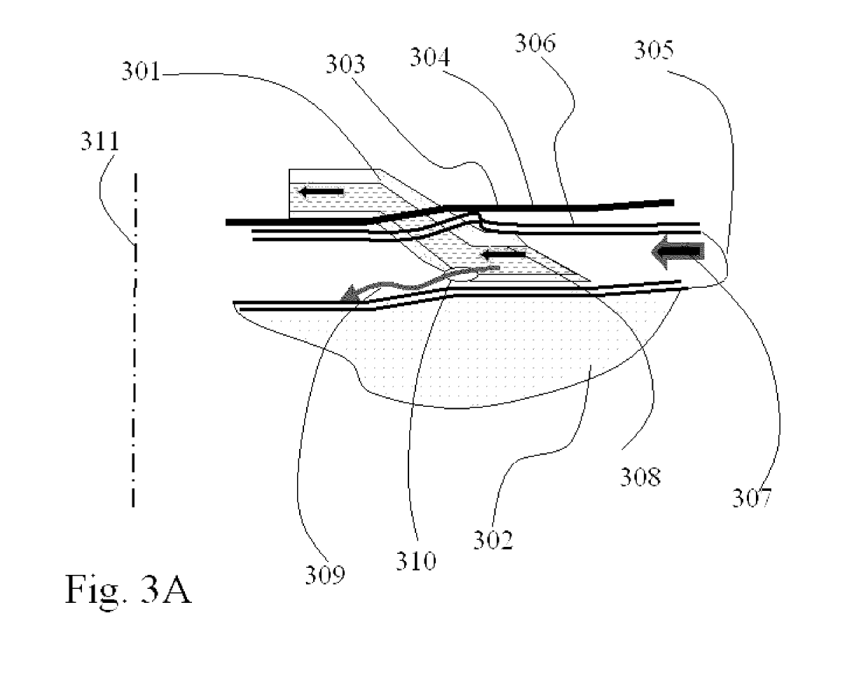

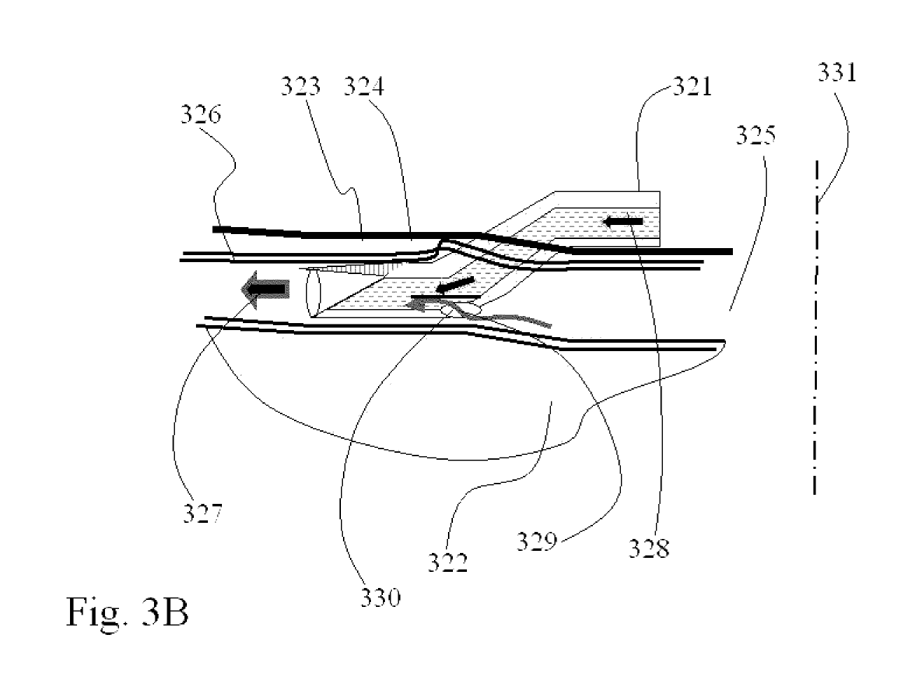

FIG. 3A—New plastic flexible dialysis tube inserted in the fistula in intake position and bent into the “dog-leg” position

301—The AV Fistula needle outside sheath of flexible dialysis tube with the needle extracted

302—Tissue

303—Skin

304—Subcutaneous tissue

305—Blood vessel

306—Blood vessel wall

307—Blood flow

308—Blood flow passing outward through the needle

309—Blood flow passing forward through the needle bypass hole

310—AV Fistula tube hole for residual blood pass through

311—Symmetry line followed by the mirrored image for blood exhaust in the blood vessel tube.

FIG. 3B—New flexible dialysis tube inserted in a blood vessel in exhaust position and bended in the “dog-leg” position

321—The AV Fistula flexible dialysis tube with the needle extracted

322—Tissue

323—Skin

324—Subcutaneous tissue

325—Fistula lumen

326—Fistula wall

327—Blood flow

328—blood flow passing inward through flexible dialysis tube

329—Blood flow passing through flexible dialysis tube bypass hole

330—AV Fistula flexible dialysis tube hole for residual blood pass through

331—gap to other.

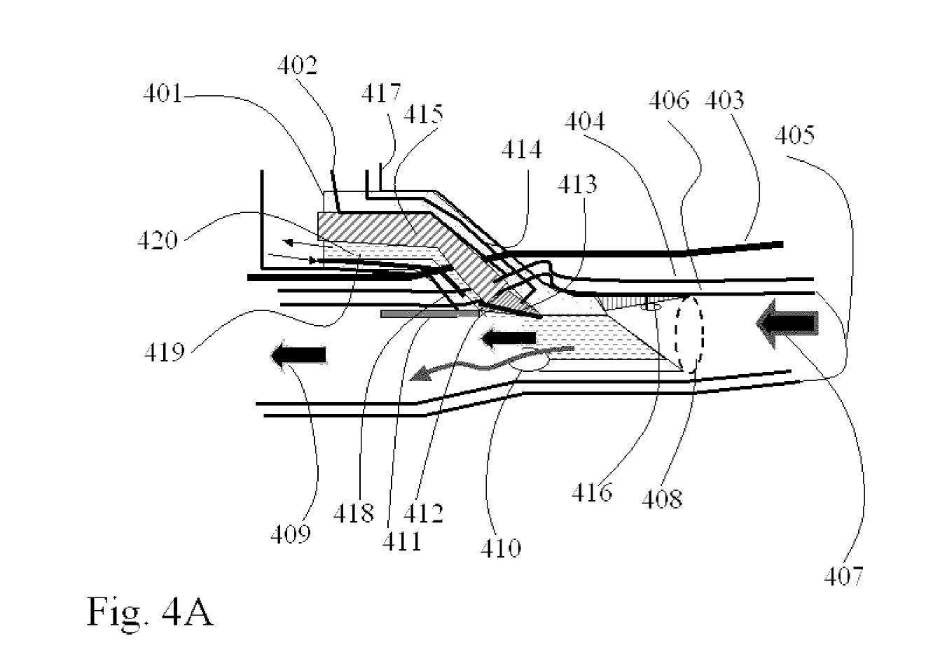

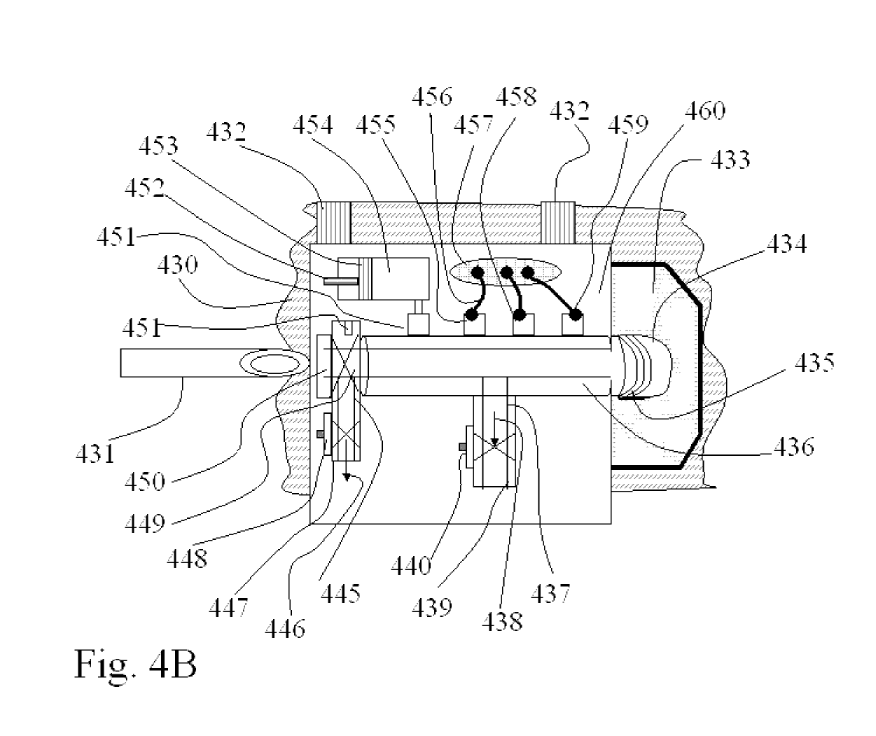

FIG. 4A—Longitudinal section of the long-term AV Fistula flexible dialysis tube with blood sensing and cleaning fixtures.

401—The AV Fistula tubing sheath with the needle extracted

402—Bladder filling micro-tubing

403—Skin

404—Subcutaneous tissue

405—Fistula lumen

406—Wall of fistula

407—Blood flow

408—Inlet umbrella expansion mechanism opened in the blood vessel

409—Blood flow passing through flexible dialysis tube bypass hole

410—AV Fistula tube hole for residual blood pass through

411—Pass through blood vessel seal in open position

412—Fluid actuated sampling tube valve in off position

413—Blood vessel seal's actuating bellows

414—Micro-fluidic channel to bellows actuator

415—Bladder or balloon inflated by sterile fluid

416—Micro-electronics measurement system

417—Multi-signal micro wire cable

418—Actuator valve signal micro-tube

419—Sterile fluid input for washing flexible dialysis tube which prevents contaminants from entering the blood stream

420—Residual fluid and washing fluid eliminated by balloon

FIG. 4B—The sterile protection enclosure and tubing functional control connections.

430—Skin surface

431—Needle in withdrawn position out of flexible dialysis tube

432—Bracelet holding the protection box tight on the body or other attachment method.

433—Sterile enclosure base on the body with antibacterial interface

434—Middle plastic tube penetrating the tissue

435—Middle tube connection to upper tube bellows in bent position

436—Lipper tube parallel to the skin

437—Intake tube that delivers blood from the fistula to the device outside the body

438—Intake blood flow exiting the tube

439—Standard medical coupling

440—On-off valve

445—Cleanup and sterilization tube

446—Clean-up sterilization flow where residual blood exits

447—Standard medical fitting coupling

448—On-off valve

449—Valve for penetration needle

450—Resilient sealing to prevent blood leakage

451—Valve actuator

452—Piston actuator to inflate the internal bladder.

453—Piston or diaphragm

454—Inflation fluid reservoir

455—Dual flow bellows actuator

456—Bellows actuator connector adaptor

457—Multi-contact connector board mounted to the platform

458—Multi-signal connector from the micro-electronic measurement system

459—Other signal (optical, ultrasound) connector adaptor

460—Operational platform with sealing case connected to base.

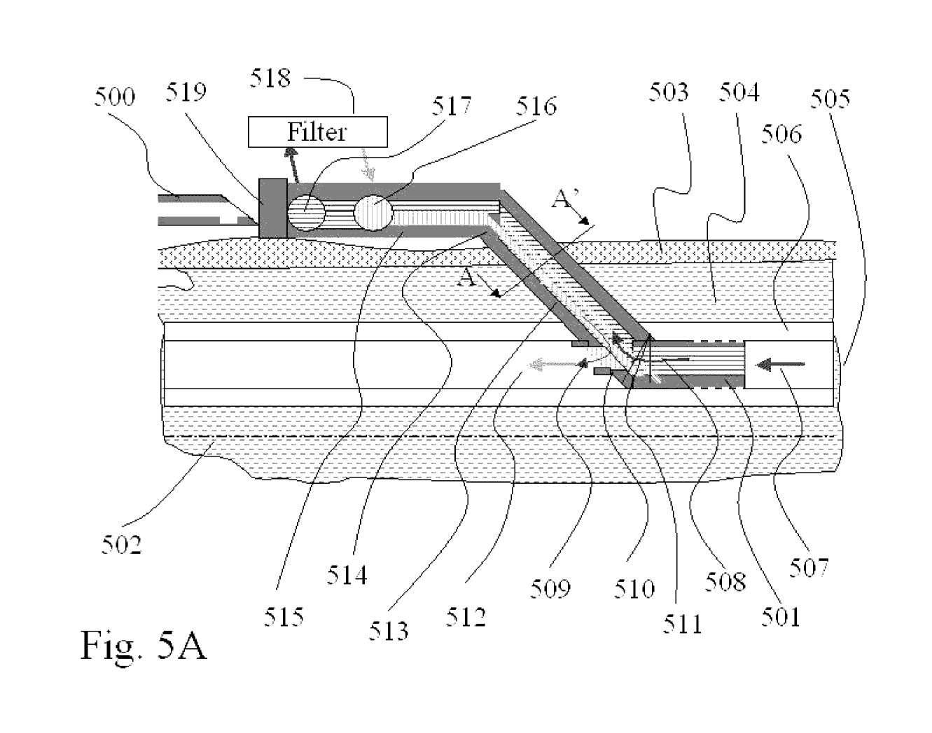

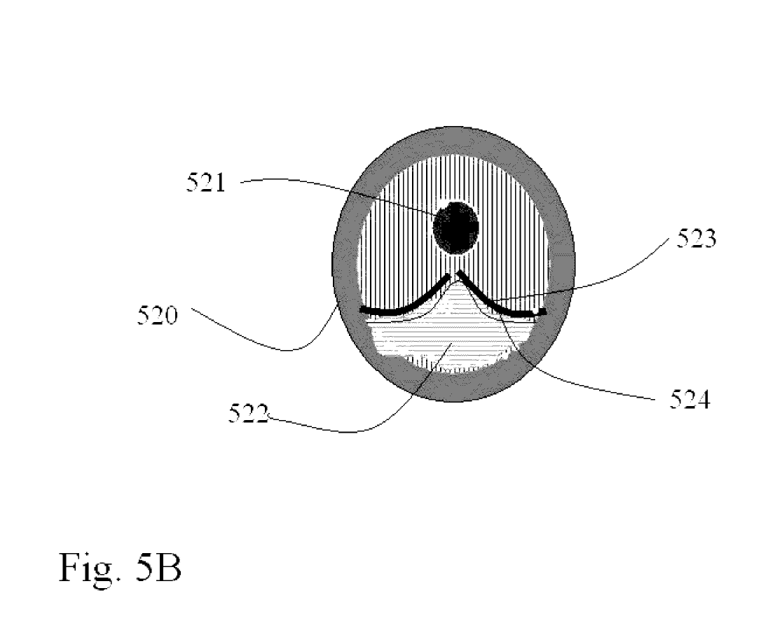

FIG. 5A—Longitudinal section through the “two in one” cannulation flexible dialysis tube.

500—The penetration needle outside sheath tube with the needle extracted

501—The umbrella structure opened in the blood vessel

502—Tissue

503—Skin

504—Subcutaneous tissue

505—Fistula lumen

506—Fistula wall

507—Blood flow

508—Blood flow passing outward through flexible dialysis tube to external device

509—Blood flow returning from outside body through the return flexible dialysis tube

510—Stoma, return hole in the fistula flowing towards the body.

511—Bellows expand here to block flow in both directions in the Off position, between dialysis sessions.

512—Venous blood flow

513—Middle zone of the plastic tube has two tubes for the blood removal and return

514—Bellows line where the tube bends forming the “dog-leg” path

515—Flexible dialysis tube outside the body attaching to the external ports.

516—External ports, standardized connectors

517—External ports, standardized connectors

518—External device

519—Terminal fitting or valve used introduce the perforating guiding needle

FIG. 5B—Cross section through the “two in one” cannulation flexible dialysis tube in the middle zone

520—The external flexible dialysis tube

521—The intake flow

522—The outtake flow

523—Semirigid hinged structure to limit the volume of the outtake flow channel

524—Semirigid membrane separating the channels

FIG. 6—Longitudinal section of the “two in one” long term cannulation flexible dialysis tube.

600—The perforation needle outside flexible dialysis tube with the needle extracted

601—The umbrella structure opened in the blood vessel

602—Tissue

603—Skin

604—Subcutaneous tissue

605—Fistula lumen

606—Fistula wall

607—Blood flow

608—Blood flow passing through flexible dialysis tube bypass hole

609—Blood flow returning from outside body through flexible dialysis tube return tube

610—Stoma, return hole in the blood vessel flowing towards the body/hart.

611—Bending expanding line where flexible dialysis tube is bellows like that expands on one side making the inner tube wall to block the input in it from the blood vessel tube.

612—Venous blood flow

613—Middle segment of flexible dialysis tube holding two tubes inside for the flow and return

614—Bellows line where the tube bends forming the “dog-leg” path

615—The final tube outside the body holding the connector fittings to the external apart.

616—Fitting For connection to external apparatus and input the blood back in the body

617—Fitting for connection the blood output to an external device

618—External device—dialyzer etc.

619—Terminal fitting used introduce the perforating guiding needle

620—Main valve—fluidic actuated

621—Dual flow separation valve in upper withdrawn position

622—Fluid channel inside the flexible dialysis tube walls

623—Elastic membrane balloon fulfilling the tubes volume with sterile liquid

624—Electric signals micro-cables from sensors and MEMS actuators

625—Dual flow separation valve actuator track

626—Pressure, temperature, flow, ph, ultrasound or optic micro-sensor array

FIG. 6B—Cross section through the “two in one” cannulation flexible dialysis tube in the middle zone in the Off position

630—Flexible dialysis tube

631—The intake flow

632—The outtake flow

633—Semirigid hinged structure to limit the volume of the outtake flow channel

634—Semirigid membrane separating the channels

635—Actuator tract of the dual fluid tract separation valve

636—Micro-tube for sterile fluid balloon actuator

637—Micro cable for microelectronics transducer

638—Multi-functional capillary tubes—for optics or fluidics

FIG. 7—The sterile protection enclosure and control for the “two-in one” flexible dialysis tube.

700—Body surface

701—Needle in withdrawn position

702—Bracelet holding the protection box tight on the body surface

703—Sterile enclosure base on the body

704—Middle plastic flexible dialysis tube penetrating the tissue

705—Middle flexible dialysis tube in bent position

706—Upper flexible dialysis tube parallel to the skin

707—Intake flexible dialysis tube that delivers blood from the fistula to the external device

708—blood flow exit port

709—Standard medical coupling

710—On-Off valve

711—Port for blood flow from the external device back into the body

712—On-Off valve

713—Standard medical coupling

714—Port for blood flow from the device into the body

715—Cleanup and sterilization tube

716—Cleaning port for blood removal and sterile fluid infusion.

717—Standard medical coupling

718—On-Off valve

719—Valve for penetration needle

720—Rubber seal to prevent blood leakage

721—Vain to close port

722—Piston actuator to inflate the internal balloon

723—Piston

724—Inflation fluid reservoir

725—Dual flow bellows actuator

726—Bellows actuator connector adaptor

727—Multi-contact connector board tight on the platform

728—Multi-signal connector from the micro-electronic measurement system

729—Other signal (optical, ultrasound) connector adaptor

730—Operational platform with sealing case connected to base.

### DETAILED DESCRIPTION

FIG. 1 Presents an actual dialysis needle in longitudinal section as it penetrates tissue and a blood vessel as is currently used, The AV Fistula needle 101 is penetrating the skin 103 and the tissue nearby 104 until it reaches the upper wall of a blood vessel 105. It is pushed forward and penetrates the nearby blood vessel wall 106 but has to go at a less acute angle inside the blood vessel to avoid further penetration through the opposite vessel wall. The blood flow 107 encounters the needle and a part passes forward through the needle 108; another part of blood flow passes forward through the needle bypass hole 109, in order to maintain active circulation and avoid coagulation. To assure this, the needle has to allow a space within the vessel to allow about 5-30% of the flow to continue past it. For dialysis purposes the extracted blood is processed in a filtering machine, 114 and another needle 112 is used to return it to the blood stream, 117 also allowing for sonic passage of blood around it.

The needles will be placed one after another on the same blood vessel leaving a gap 111 between. The second needle 112 that puts blood back in the vein is punched in the opposite direction making possible that the blood coming from the external device 114 (dialysis or analysis, or other) that takes the blood from the first needle through the input tube 115 and after processing places it at the output tube 116 and returns in the second needle 112 in the blood flows recombination point 117, where it mixes with the blood left for vein's maintenance 110.

The process just described, requiring 2 punctures and 2 needles for each session of hemodialysis is traumatic for patients, particularly for their arterio-venous shunts. The needles are typically discarded as bio-hazardous waste after only one use. In this conventional process, the patients' distress and their risk for vessel damage and other medical complications is higher than it needs to be.

FIG. 2—shows the new AV Fistula needle's cutting end coated in the plastic flexible dialysis tube, inserted in a blood vessel, with the more compliant material inside the blood vessel, reducing the risk of vessel damage.

In this new technology the AV Fistula needle 201 has a profiled cutting edge 202 followed by a blunt edge for blood vessel's elastic stretching without cutting 203, that is meant to assure a tight contact and elastic sealing inside the blood vessel penetration hole, 205 minimizing the blood leakage. A needle bump 204 is used for plastic flexible dialysis tube umbrella opener activation.

The needle is introduced without penetrating the other side of blood vessel wall 206. Because different patients have different size blood vessels, a dynamic adjustment to the blood vessel diameter is made by using an umbrella opening structure inside the vessel 208 that is activated by an inner bump 209 which, when pressed by the needle bump 204 triggers the breaking of a plastic seal 210. The umbrella structure then opens inside the blood vessel to get gently tight against the walls, to form a seal.

The new tube that surrounds the penetration needle is a biocompatible plastic tube 211 that is less stiff than the steel needle and harmless to the blood vessel.

This feature replaces the steel needle of previous technologies with a soft plastic tube that maintains its profile and assures the maximum flow of blood. It may be made from biodegradable polyamide, but may as well be made of any type of blood and vessel compatible plastic.

FIG. 3A shows a new plastic flexible dialysis tube inserted in a blood vessel in intake position and bent in the “dog-leg” position which is another embodiment of the present invention. It is known that any bending in a fluid tube is lowering the flow limit where laminar to turbulent flow transition occurs, which in case of blood may damage the cells but, in this case with mild radius and low flow the effects are minimal. The puncturing needle is then extracted from the sheath flexible dialysis tube 301 that remains in the blood vessel 305. Illustration shows it penetrates skin 303, the subcutaneous tissue, 304 and the upper blood vessel wall 306 remaining sealed inside in the blood flow 307 of the fistula. Blood flow passing forward through the noodle cover 308 that is sealed tight in the blood vessel prevents any leakage in the tissue 302. In order to prevent blood flow stagnation and deterioration a percentage of blood flow passes forward through flexible dialysis tube bypass hole 309 and the rest goes forward through the AV Fistula lumen 310. A similar flexible dialysis tube operates to reinfuse the purified blood at the return site.

FIG. 313 shows a new plastic flexible dialysis tube, as embodied in the present invention, inserted in a blood vessel in exhaust position and bent in the “dog-leg” position.

The AV Fistula sheath tube with the needle extracted 321 is implanted through the skin 323 and subcutaneous tissue, 324 in the blood vessel 325 penetrating the blood vessel tube wall 326. The blood flow 327 is reconstituting at the nominal level by joining the blood flow passing forward through flexible dialysis tube 328 and blood flow passing forward through flexible dialysis tube bypass hole 329. In order to prevent blood clot formation a hole is provided for residual blood pass through 330.

FIG. 4A shows a longitudinal section through the long-term AV Fistula flexible dialysis tube with blood cleaning fixtures. In order to reduce the harm inflicted by repeated perforations, plugs are developed to enable long-term use. They are built from special plastic materials, or possibly titanium coated to produce a minimal negative interaction with the tissue, and prevent tissue buildup. Studies would be needed to evaluate factors of patient preference and relative safety or multiple punctures versus the long-term, multiple use connector box attached to the patient's body.

The AV Fistula flexible dialysis tube with the needle extracted 401, showing the plastic flexible dialysis tube fitted on the body surface in the position to be sealed and have blood flow directly through it. To stop the blood flow in the tube a bladder is inflated with a sterile fluid, and gently removing the blood in the tube in such a manner that no blood will remained trapped between the inflating bladder or balloon 415 and the tube wall 401. Bladder filling micro-tubing 402 takes the sterile liquid from a syringe and inflates the balloon to fulfill the tube's volume.

Flexible dialysis tube is left in the position that penetrates the skin 403 and subcutaneous tissue 404 into the blood vessel 405, puncturing only one blood vessel tube wall 406—sufficient to collect the entire blood flow 407—because while penetrated inside an input umbrella expansion mechanism opened in the blood vessel 408 is activated, opening tight to the blood vessel walls 406.

In dual-flow operation, valve 412 occludes the lumen so that no blood flow passes through flexible dialysis tube bypass hole 409. When the device is in Off mode, valve 412 is covering the outflow passage so that all the blood passes directly through the hole 410, resulting in a minimal dynamic pressure drop around the inserted tube.

Immediately after the seal closes flexible dialysis tube, the blood cleanup procedure starts by pumping more sterile fluid into the flexible dialysis tube's bladder or balloon 415, while sterile fluid for washing dean flexible dialysis tube 419 is pumped in. Because this device is inserted in a blood vessel lumen, a microelectronic measurement system 416, generically called MEMS devices, can be a part of the device, enabling measurement of many types of signals through a multi-signal micro wire channel 417.

The actuator valve for the micro-tube 418 may use the same sterile liquid to inflate or dis-inflate the actuating bellows, or may use electric signal and a MEMS device as actuator.

During the sealing procedure the pump is introducing sterile liquid through the micro-tube 419 that is injected immediately after the valve 412 making a volume of liquid flowing between the inflating balloon/bladder 415 and flexible dialysis tube wall 401 to contain less and less blood traces up to the moment is completely clean and the balloon fills all the volume, making the residual volume 420 be a minimum.

FIG. 4B—The sterile protection enclosure and needle-tubes control system. The desired outcome is to reduce the number or punctures of the blood vessel and to use one penetration for more than 1 week, with a high level of patient comfort and safety.

This requires a protection device attached to the patient's skin to keep the in-dwelling flexible dialysis tube sterile and free of any mechanical stress, ready to be connected to the dialysis machine and start the process immediately. In engineering we call such a device a connection box that will sit on the body surface 430 over the puncture zone 431. A bracelet-like device 432 holding the protection box tight on the body part or limb is connected to a sterile enclosure 433 on the body surface. The middle plastic tube penetrating the tissue 434 is maintained in the position with middle tube connection to upper tube bellows in bent position 435 and upper tube 436 is parallel to the skin, holding the intake tube that delivers blood from the blood vessel to the device outside the body 437. Blood flow exiting the tube 438 via a universal medical coupling 439. Each flexible dialysis tube has an On-Off valve 440 used to seal the tube after cleaning with sterile liquid after its disconnection from the external apparatus. Reconnection can be made without introducing air bubbles.

Cleanup and sterilization tube 445 enables sterilization flow with residual blood coming out 446 and a standardized medical fitting 447 and an on-off valve 448. The tissue penetration needle pass-through valve 449 has any leakage restricted by a rubber seal. 450,

Other connectors include valve actuator 451, a bi-directional piston actuator 452 to inflate/deflate the internal balloon, 453 from an inflation fluid reservoir 454 holding less than 1 ml. of sterile liquid.

The dual flow bellows actuator 455 has a connector adaptor 456.

The electronic measurement system embedded in the flexible dialysis tube has a multi-signal connector from the electronic measurement system 458 which carries the signal to a multi-contact connector board on the platform, 457 that may also include other signal (optical, ultrasound) connectors 459.

The entire operational platform 460 with sealing case connected to base, is sealed tight preventing septic infiltration or mechanical stress to the tube.

FIG. 5A—Longitudinal section through the “two in one” cannulation flexible dialysis tube that allows for the blood to be extracted and introduced through a single puncture, rather than by using two punctures as described above.

The AV Fistula flexible dialysis tube, outside sheath tube 500 with the needle extracted has the umbrella structure opened in the blood vessel 501 making it tight to the walls. The intermediary segment is penetrated through the skin 503, and the subcutaneous tissue 504 into the blood vessel 505 surrounded by the tissue 502. The penetration is done in such a manner that only one blood vessel tube wall 506 to be perforated allowing the blood flow 507 to be entirely collected and blood flow is passing forward through the needle 508 going to the standardized connector. The blood flow returning from the dialysis machine through the needle return tube 509 where it has an opening made by a stoma, return hole in the blood vessel flowing towards the body core/heart 510.

Bent expanding line 511 where the flexible dialysis tube is bellow like that expands on one side making the inner tube wall to block the input of blood.

Venous blood flow 512 is passing through middle zone of the plastic tube 513 with its two channels inside for the blood removal and return.

The fitting for connection to external apparatus 516 for pumping the blood back in the body; a fitting for connecting the blood output 517 to an external device 518; and a terminal fitting 519 used introduce the perforating guide needle.

FIG. 5B—Cross section through the “two in one” cannulation tubing middle zone. It shows the external plastic tubing 520 that contains the intake flow 521, the outtake flow 522 which contains a semi-rigid structure to limit the volume of the outtake flow channel 523. The elastic semi-rigid membrane separating the channels 524 to allow a large aperture for the intake flow and let a reasonable but adequate passage for the blood return coming from the external apparatus.

FIG. 6 Cross section through the “two in one” long term cannulation flexible dialysis tube that is closed when the apparatus is disconnected from the body.

The insertion flexible dialysis tube 600 with the needle extracted is shown. The upper skin layer 603, the subcutaneous flesh 604, and the fistula wall 606 which are perforated by the needle. When the needle 600 which made the perforation is extracted, the umbrella structure 601 is opened in the fistula 605 preventing any leaks into the tissue 602 and collecting all the blood flow 607 making the blood flow through flexible dialysis tube's lumen 608. In the Off position it passes straight through the stoma 610, returning towards the body core/heart 609.

In order to accommodate the dogleg shape the bend 611, where the flexible dialysis tube has bellow-like membrane that expands to form 2 channels, with the second channel 612 for return flow.

Middle zone of the plastic tube holding two channels inside for the blood flow and return 613. Bellow membrane extends through the “clog-leg” segment, 614. Segment of tubing outside the body, 615, making connection to external apparatus, 616 and 617 (for blood ports to an external device 618 that can be a dialysis machine, etc). Another terminal fitting 619 is used to introduce the perforating guiding needle that is used when the perforation is made, and a blunt device, when the tube is removed from the patient at the end of its use.

Valve attached to the bellows semi-rigid membrane 620, 621 directs blood flow into the dual-lumen channels or bypass channel 608.

To purge residual blood from fluid channel a micro tube runs inside its wall 622 which inflates an elastic membrane balloon with sterile liquid 623. Electrical micro-cables 624 come along the tube wall carrying the signals from the micro-sensors 626, that can measure blood pressure, temperature, flow, pH, etc. Such micro-cables could also control the separation valve actuator 625 if it were to be operated by a MEMS (micro-electronics mechanical system).

FIG. 6B shows a cross section through the “two in one” cannulation flexible dialysis tube in the AA′ zone when the tube is in “stand-by” mode between perfusion/dialysis sessions, that allows keeping the tube inserted in the body safely, and reduce the risk of repeated punctures. This adds some patient discomfort (from the connection box being constantly attached to the body), but reduces the risk and discomfort of repeated fistula punctures.

The main plastic flexible dialysis tube 630 in the Off position has its main lumen 631 occupied with a balloon inflated with sterile liquid. The blood return channel 632 is now compressed while its lumen is washed with sterile fluid. The inflated balloon 633 compresses the semi-rigid elastic membrane 634 against the tube wall. Running inside the tubing wall we see the micro-tube 635 for bellows expansion, another for balloon filling with sterile fluid 636, a micro cable for micro-electronics measurement array 637, other multi-functional capillary tubes—for optics or fluidics 638.

FIG. 7 shows the sterile protection enclosure and functions control for the “two-in one” tube.

The body part or hand zone 700 is shown with the needle 701 in withdrawn position out of plastic cover tubes, but along the external tube segment axis.

A bracelet 702 holding the protection box tight on the body part or limb and its sterile enclosure base 703 attaching to the body.

The flexible dialysis tube 704 penetrating the tissue showing tubing bend 705 and the upper tube parallel to the skin 706.

Standardized fitting, 707, 711, 715 clearly depicted on the outer cover of the connection box, makes all the connections to facilitate patient interchange and connections to various medical devices.

The intake tube 707 that delivers blood from the blood vessel to the device outside the body and intake blood flow exiting the tube 708 have universal/standardized medical coupling 709 followed by On-Off valve 710 controlling blood outflow to the external device. The return port 711 for blood return also has an On-Off valve 712, and the standardized medical coupling 713 for the blood flow from the external device back into the body 714.

To make a safe easy procedure a cleanup and sterilization tube 715 allows sterilization fluid to be injected and residual blood to be removed 716. Standardized medical coupling 717, on-off valve 718 and a penetration needle pass through valve 719 equipped with rubber sealing to prevent blood leakage 720. The tube switching from active mode where blood flows out and in through the tube back in the blood vessel is done using two inner channels that are opened by outside valve actuators 721 that may use a fluidic or electric actuation.

The clean, residual blood-free blocking of the middle segment of the plastic tube is done using a sealed piston actuator 722 to inflate the internal balloon, using a piston 723, inflation fluid reservoir 724, and an actuator (not shown). The dual flow bellows actuator 725 is connected at a bellows actuator connector 726 on a multi-contact connector board attached to the platform 727 that may directly control the bellows. An adaptor module inside the connection box (not represented for clarity purposes), may use an external signal for controlling the tube modes: active, preparing to close and washing, closed and prepare to open.

To make the operation more controllable, a multi-micro-sensor array may be is inserted in the input and output stoma of the tube, giving a plurality of physiologic signals difficult to be accessed by other means. These signals are transported to a multi-signal connector from the electronic measurement system 728 by the appropriate connectors. Other signals' (optical, ultrasound) connector adaptor 729, may be directly accessed by direct or wi-fi connection to external measurement devices. Then connection box case is connected to base 730.

The procedure contains the following steps:
1. With the penetration needle inserted inside the plastic flexible dialysis tube the perforation of the blood vessel and cannulation is performed.
2. The perforation needle is withdrawn and the external flexible dialysis tube is bent forming the “dog-leg”, and the stoma valves are triggered to open, by breaking the inner locking micro-seals.
3. The sterile platform is stuck on the skin around the perforation site and sterilized. The connectors and actuators are installed.
4. A test actuation is done and body parameters measurement tested.
5. The external devices are connected and the tube is set on operational mode.
6. After ending the procedure, “prepare to close” mode is ordered and the separation valve is set to off, and the closing valve is set to on, making the blood bypass the flexible dialysis tube inside the fistula. The cleanup procedure starts by inserting sterile liquid simultaneously with slowly inflating the balloon, until it fills the inner volume of the tube so that no blood or sterile liquid remains inside. The procedure is finished when these are accomplished.
7. The electronic measurement devices remain on or off depending on user's need.
8. When the next dialysis, perfusion, or infusion is needed the “prepare to open” is ordered and the balloon is evacuated and withdrawn from its position against the tubing wall, the shutter valve is opened while the separation valve is on, making the apparatus ready for connection to external machines. The input and output ports may be opened and the active mode is set to On, by fully actuating the inner separation valve.
9. The infusion or blood extraction or dialysis exchanges then take place in active On mode until the end of operation when the “prepare to close” and “passive/closed” mode is set to On.
10. The cycle 5-9 may be repeated several times as necessary or as long it is safe for the patient the duration of maintaining the inserted tube in the fistula or other vessel will be established by medical need. Then the flexible dialysis tube is extracted by withdrawal, or if necessary by reintroducing a flexible obturator device to facilitate removal.
11. If only single use flexible dialysis tubes are used, comprising the stages 1,2 and 10, the operation is simpler, as they would not use the various flow directing valves or cleaning balloon additions in the more advanced devices proposed above. These devices would require the same precautions as current dual needle techniques placement to avoid blood mixing, keeping adequate residual flow in the shunt, proper diameters to assure good flow, etc.

When flexible dialysis tube is not in use, blood ports are sealed by valves and various materials may be used in the connection box Or over the connection box to keep sterility. These would include foils of metal or plastic or gaseous additions.

### BRIEF DESCRIPTIONS OF INVENTION

The present invention refers to a set of improvements to the actual technique and apparatus of perfusion and dialysis having several stages of application that are not mutually exclusive.

The main embodiment of the invention refers to the enhancement of the perfusion needle by adding a special plastic flexible dialysis tube covering the needle. The stiff needle is used for penetration and to insert flexible dialysis tube that will remain inside and shape itself to the vessel, while the stiff needle is extracted. Flexible dialysis tube has a structure that opens gently inside the blood vessel, preventing blood leaks from the vessel and bends along the body parallel with the skin to minimize patient distress.

It is possible to reduce the number of perforations for a dialysis session from two to one by using a two in one flexible dialysis tube installed over a guiding needle. After it's in the vessel and the needle is withdrawn, flexible dialysis tube opens forward and backward and the initial tube becomes a dual function tube by the opening of a supplementary partition inside, so the blood comes out using one partition and is pumped back in the blood vessel using the secondary partition.

Using advanced technologies, a controllable blood extraction/perfusion flexible dialysis tube may be developed which once inserted in the body may be safely maintained there for long periods, assuring it remains sterile and safe to use as a fluidic connector. One key issue is that blood that remains static in flexible dialysis tube may coagulate or deteriorate. In order to eliminate this possibility all the residual blood from a closed tube is eliminated by the help of another bladder placed on the other internal surface of the tube that may be inflated at will removing any blood or liquid trapped inside the dead-end tube.

To further improve this process, blood is cleared by purging with a sterile liquid. Further, the use of embedded micro-electronics and micromechanics placed as a sensor array inside the tube could measure blood pressure, temperature, blood composition and chemical parameters, data that normally require multiple devices and blood removal to acquire. Measurement of flow and pressure inside the patient's AV shunt could possibly enable tuning of dialysis pump parameters for an optimal physiologic result.

Further, the present invention proposes a connection box that would be attached to the patient's surface so that he could be connected in seconds to an external blood processing or infusion device, and through which physiologic measurements could be made continuously or as desired.

### Examples of the Invention

Thus it will be appreciated by those skilled in the art that the present invention is not restricted to the particular preferred embodiments described with reference to the drawings, and that variations can be made therein without departing from the scope of the present invention as defined in the appended claims thereof. The present invention consists in the development of a set of improved vascular access devices that could be used for cannulation and blood removal or reinfusion, or the introduction of any fluids to the circulatory system of the body for humans and animals, in customized versions, regarding gauge, length and functionalities.

The application of these customized versions will extend the range of multiple usage minimizing the negative impact of the treatment on patients, and also reducing undesired collateral effects and medical complications. The use of the embedded sensors will bring progress to the practice of medicine, allowing the patient's blood pressure, temperature, flow, composition of the blood and its chemical properties to be monitored continuously and used in diagnosis and equipment control. Some derivatives of this equipment, without the function of blood and fluid transfer might be developed as implants for measurement purposes only. The application of the present invention will generate a step forward in medicine, by intensively using multi-parameter monitoring and more body-friendly invasive devices.

What is claimed is:

1. A dialysis catheter device tube sheath assembly comprising:
a. A guiding-perforation needle;
b. A dialysis catheter flexible tube sheath that is covering the perforation needle comprising:
i. Three bendable segments where:
1. first segment is inserted with a needle in a blood vessel and remains there after needle's withdraw and contains:
a structure that opens counter-blood flow on the blood vessel made of: i. a plurality of stiffer plastic fibers where each fiber has a bump pushed when needle is taken-out and makes fiber open; ii. a thin foil/membrane that connects plastic fibers and get in contact with blood vessel's wall sealing on it;
a tube that hold: i. an opening structure being sealed into it; ii. a set of electronic sensors to measure: 1. blood pressure; 2. blood flow; 3. temperature; 4. electric conductivity; 5. chemical content; 6. means of transmitting the signals to outside measurement equipment; iii. a bending structure that connects this segment to a middle segment comprising: 1. a rigid structures that prevent tube squeezing connected on the smaller bending radius side, where signal transmission structures pass through; 2. a plastic, expandable membrane that seals inside the structure; 3. an opening structure that has two states: a. a small hole, that allows a residual small blood flow pass through along the blood vessel, during operation; b. a large hole that allows the entire blood flow to pass through in the period between operations, forming a seat for a valve that is placed over to occlude the hole during operation and leave only the small hole open, where that valve and its actuator is placed on the second segment; c. a soft opening structure along blood vessel made of a soft membrane opening and entering in contact with blood vessel's wall, sealing on it, pushed large by the exhausted blood flow, and prevents blood vortex and recirculation;
2. a second segment on which the punctured and stretched blood vessel tube seals on its outside surface comprising:
a valve that can occlude either: i. a first segment hole making blood flow through the second segment, or; ii. a second segment, allowing blood pass through first segment in the time interval between two consecutive operations of blood extraction;
valve's actuator, that moves the valve between, in desired positions;
means to carry power through the second tube to actuate the valve, where the actuator may be: i. hydraulic when actuated with a blood compatible fluid, and requires micro-tubes to carry fluid along the second segment tube; ii. electric when actuated with a micro-mechanic electric device (MEMS) and requires electric micro-cables; iii. means to control the good operation of the valve;
a separating wall along the tube ending with the valve joint and valve actuator that divides the tube's space in two channels: i. a larger channel used to take out blood moved by its own pressure; ii, a smaller channel used to introduce back the blood moved by a pump pressure;
where the separating wall carries means for valve actuating as fluid micro-tubes and micro-cables;
a membrane sealed on the border of the walls of the tube used to take blood out, that is actuated with fluid, and when: i. fluid is pushed inn inflates the membrane forming a balloon that occludes blood channels firmly stopping the flow, by pressing on the separation wall and squeezing it on the segment tube wall; ii. fluid is extracted the balloon squeezes on the tube's wall, leaving the tube open for blood flow;
means to carry fluid to the membrane, made of micro-tubes;
a set of micro-tubes ending with exit at lower border of membrane, near bending structure used to introduce a liquid to remove the blood before occluding the blood channels;
a bending structure outside tissue and skin that connects to third segment and contains: i. a rigid structure meant to maintain the shape and prevent the structure to fall inside and squeeze; ii. an elastic membrane that seals on rigid structure maintaining two channels separated and structure sealed; iii. passage of tubules and cables through the smallest curvature section;
a third segment outside the body, sealed on the second segment, aligned along patient's skin that is used to separate actions and functions comprising;
a longitudinal section ended with a valve, that is: i. open when perforation, rigid needle is in, and ii. shuts when needle is extracted;
lateral on longitudinal section are blood channels outputs ending with: i. valve: 1. for blood extraction; 2. for blood return; ii. after valve blood tube connection and; iii. near valve micro-tubes for air extraction prior to valve opening for operation;
entries and exits for electric cables and micro-tubes for connection to functional devices as: 1. actuators for: 2. valve; 3. balloon; ii. measurement instruments for blood parameters, and tube functionality; iii. liquid insertion and extraction from the tube;
ii. A connection box to ease the access comprising:
1. a base that is placed on the limb using;
straps;
glue or sticky surface that sticks on skin;
2. an elastic fixture on base of flexible tube's third segment, allowing that connectors to be rigid on base;
3. a lid that seals base making it antiseptic;
4. a set of actuators and a control unit inside box comprising:
a valve actuator;
a balloon actuator;
a liquid introduction in flexible tube;
a liquid connector air extraction;
electronics for: i. measuring the parameters from sensors and; ii. interface with external apparatus; iii. control actuators;

2. (canceled)

3. (canceled)

4. A perfusion-dialysis needle-flexible-tube sheath assembly according to claim 1 where perforation steel tube placed inside flexible tube has a bump that protects plastic flexible tube at entry and when withdrawn from the flexible tube after penetration inside blood vessel opens flexible tube front end inside blood vessel.

5. A perfusion-dialysis needle-flexible-tube sheath assembly according to claim 1 where that uses the hydrophobic, anticoagulant and antiseptic coating.

6. A perfusion-dialysis needle-flexible-tube sheath assembly according to claim 1 where needle has a sharp edge to perforate into tissue followed by a blunt edge to stretch tissue open, and a bump to protect edge of flexible tube and set open its intake part.

7. A perfusion-dialysis needle-flexible-tube sheath assembly according to claim 1 where shutter valves that are stopping blood flow along blood vessel in and out flexible tube, are hydraulically actuated by a piston with liquid and inflatable bellows forcing blood flow through flexible tube to external apparatus.

8. A perfusion-dialysis needle-flexible-tube sheath assembly according to claim 1 where flexible tube contains a diaphragm inside that separates two blood paths each path having an adjustable cross section.

9. A perfusion-dialysis needle-flexible-tube sheath assembly according to claim 1, where another sealed diaphragm creates a balloon inside flexible tube that is connected to a piston containing a liquid that inflates balloon and closes flexible tube in shut position, eliminating the residual blood.

10. A perfusion-dialysis needle-flexible-tube sheath assembly according to claim 1, where a flexible tube is connected to a hydraulic assembly featuring fast, air bubble-free connection.

11-20. (canceled)

21. A perfusion-dialysis needle-flexible-tube sheath assembly according to claim 1, where the plug is designed to occupy tube's inner volume, and seal it in sectors, preventing bleeding.

22. A perfusion-dialysis needle-flexible-tube sheath assembly according to claim 1, where tube is inserted using a perforation needle that opens and releases flexible tube inside the blood vessel, flexible tube, which is then bends to conform and align to the tissue shape.

23. A perfusion-dialysis needle-flexible-tube sheath assembly according to claim 1, where external pump is synchronized with the patient's pulse using the embedded sensor system in order to reduce pressure difference and their adverse turbulence effects.

24. A perfusion-dialysis needle-flexible-tube sheath assembly according to claim 1, where an amount of fluid volume extracted during the session is controlled by the electronic measurement system, helping the patient to leave dialysis session with optimal intra-vascular volume.

25. A perfusion-dialysis needle-flexible-tube sheath assembly according to claim 1, where embedded electronic and optical sensors are used to monitor patient's vital parameters during procedures, and using collected data to optimize patient's treatment.

26. A perfusion-dialysis needle-flexible-tube sheath assembly according to claim 1, where plastic tubing has ends which expand in blood vessel, sealing to its walls where expanding structures are made of various stiffness plastic materials that will give optimal pressure on walls to seal and prevent any leakage or back flow.

27. A perfusion-dialysis needle-flexible-tube sheath assembly according to claim 1, where tube is connected to a connection box for switching tubing function from its On to its Off position.

28. A perfusion-dialysis needle-flexible-tube sheath assembly according to claim 1, where connector box has two pistons electrically actuated, that inflate and deflate shutter valve actuates bellows and inflates cleaning balloon.

29. A perfusion-dialysis needle-flexible-tube sheath assembly according to claim 1, where connection box uses several layers of containment made of various composite materials, plastics, metals, and gas layers, to assure sterility or antiseptic protection and mechanical protection.

30. A perfusion-dialysis needle-flexible-tube sheath assembly according to claim 1, where protection box contains electronic measurement and transmission systems for patient's vital parameters.

31. A perfusion-dialysis needle-flexible-tube sheath assembly according to claim 1 where flexible tube and protection box may be hit from outside, squeezed without inflicting more tissue damage.

32. A perfusion-dialysis needle-flexible-tube sheath assembly according to claim 1 that uses a connection box applied on tissue and sealed on, covering the flexible tube exit from skin and offering an antiseptic protection to penetration zone.
